(19) United States
(12) Patent Application Publication
(10) Pub. No.: US 2003/0207272 A1
(43) Pub. Date: Nov. 6, 2003

(54) Composition and methods for detection of von willebrand's disease

(76) Inventor: Mary Whitely, Rockville, MD (US)

Correspondence Address: Ivor R Elrifi, Mintz Levin Cohn Ferris, Glovsky & Popeo, One Financial Center, Boston, MA, 02111, US

(21) Appl. No.: 10/088,256

(22) PCT Filed: Apr. 7, 2001
(86) PCT No.: PCT/US01/11487

(51) Int. Cl. C12Q 1/68; C07H 21/04; C12P 21/02; C12N 5/06

(52) U.S. Cl. 435/6; 435/69.1; 435/320.1; 435/325; 536/23.2

(57) ABSTRACT

The sequence of part of intron (42), the complete sequence of exon (42) and part of intron (43) of the canine von Willebrand's Factor cDNA and deduced amino acid sequence is provided. Also provided are part of intron (6), the complete sequence of exon (7) and part of intron (7). The mutation which causes von Willebrand's Disease in Doberman pinschers, Shetland sheepdogs, Manchester terriers and Poodles are also provided. Methods for diagnosing a canine as clear, carrier or affected for von Willebrand's disease are provided.

### RELATED U.S. APPLICATION

This application claims priority to U.S. S No. 60/195,544 filed Apr. 7, 2000 which is incorporated herein by reference in its entirety.

### FIELD OF THE INVENTION

The present invention relates generally to compositions and methods for detection of von Willebrand's disease. In particular, the present invention relates to the use of nucleic acids and polypeptides associated with the von Willebrand's Factor gene in the diagnosis, treatment and prevention of von Willebrand's disease in dogs.

### BACKGROUND OF THE INVENTION

Von Willebrand's disease (vWD) is the most common bleeding disorder in both humans and dogs and may vary in severity due to the of levels of von Willebrand's factor that results from a quantitative or qualitative defect in von Willebrand's Factor (vWF) (Ginsburg, D. et al., Blood 79:2507-2519 (1992); Ruggeri, Z. M., et al., FASEBJ7:308-316 (1993); Dodds, W. J., Mod Vet Pract 681-686 (1984); Johnson, G. S. et al., JAVMA 176:1261-1263 (1988); Brooks, M., Probl In Vet Med 4:636-646 (1992)). vWF functions to stabilize Factor VIII (hemophilic factor A), and aids in the adhesion of platelets to the subendothelium, allowing them to provide hemostasis more effectively. Patients, whether humans or dogs, may bleed severely if they have a defective vWF or if they lack vWF entirely.

The nomenclature for vWD is based on the severity of the disease which is genetically and clinically heterogeneous. There are three clinical types, called 1, 2, and 3 (formerly I, II, and III; see Sadler, J. E. et al., Blood 84:676-679 (1994) for nomenclature changes), Type I vWD appears to be caused by a reduced level of vWF and is generally inherited in a dominant, incompletely penetrant fashion. It is considered to be a mild form of the disease, and is the most common form of vWD found in most mammals, but can still cause serious bleeding problems.

In Type 2 vWD, patients may have essentially normal levels of vWF, but the factor does not function properly. (Ruggeri, Z. A, et al, FASEB J 7:308-316 (1993); Brooks, M., Probl In Vet Med 4:636-646 (1992)). Type 2 is inherited in a dominant fashion and is not common in dogs (Turrentine, M. A, et al., Vet Clin North Am Small Anim Pract 18:275 (1988)).

Type 3 vWD, the most severe form of the disease, is inherited as an autosomal recessive trait and affected individuals have no detectable vWF in their blood. This results in serious bleeding episodes that require transfusions of blood or cryoprecipitate. Heterozygotes have moderately reduced factor concentrations, but can clot normally.

Because homozygotes have no detectable vWF they have a severe bleeding disorder. Although heterozygotes have reduced levels of the factor, they are clinically normal (Brooks, M. et al., JAVMA 200:1123-1127 (1992)). Scottish terriers have Type 3 vWD (Dodds, W. J., Mod Vet Pract 681-686 (1984); Johnson, G. S. et al., JAVMA 176:1261-1263 (1988)). The prevalence of vWD among Scottish terriers is estimated to be from 27-31%. This included both homozygotes and heterozygotes for the mutant allele (Stokol, T. et al., Res. Vet. Sci. 59:152-155 (1995); Brooks, M., Proc. 9th ACVIM Forum 89-91 (1991)).

The current method of detection of affected and carriers dogs is done by vWF antigen testing (Benson, R. E. et al., Am J Vet Res 44:399403 (1983); Stokol, T. et al., Res. Vet. Sci. 59:152-155 (1995)) or by coagulation assays (Rosborough, T. K et al., J. Lab. Clin. Med. 96:47-56 (1980); Read, M. S. et al., J. Lab. Clin. Med. 101:74-82 (1983)). These tests can yield variable results, and can be influenced by such things as sample collection, sample handling, estrous, pregnancy, vaccination, age, and hypothyroidism (Strauss, H. S. et al., New Eng J Med 269:1251-1252 (1963); Bloom, A. L., Mayo Clin Proc 66:743-751 (1991); Stirling, Y. et al., Thromb Haemostasis 52:176-182 (1984); Mansell P. D. et al., Br Vet. J. 148:329-337 (1992); Avgeris, S. et al., JAVMA 196:921-924 (1990); Panciera, D. P. et al., JAVMA 205:1550-1553 (1994)). Thus, breeders may need to have the tests done several times in order to determine the status of their animal with respect to vWD.

A more useful test would be a direct DNA test using sequences from the canine vWD gene. Since purebred dog populations are highly inbred, most disease causing mutations are the result of a founder effect, and are therefore conserved within a given breed. Therefore, it would be desirable to provide specific mutations for specific breeds. Furthermore it would be desirable to provide a method for determining the genotype of an animal so that carriers of the mutation could be bred to those who are clear of the mutation, and therefore no affected offspring would be produced

### SUMMARY OF THE INVENTION

In one aspect, the invention involves an isolated nucleic acid molecule having a nucleotide sequence encoding a mutated canine von Willebrand's Factor polypeptide which causes canine von Willebrand's disease. The nucleotide sequence of this nucleic acid sequence is capable of hybridizing under high stringency conditions to the complementary sequence of the sequence of SEQ ID NO:1, having a mutation at nucleotide 172, or SEQ ID NO:2, having a mutation at nucleotide 384.

The invention also involves a vector containing the nucleic acid molecule. In a further embodiment, the invention is concerned with a cell containing such a vector.

In a still further embodiment, the mutation at nucleotide 172 of SEQ ID NO:1 is a nucleic acid substitution. By “nucleic acid substitution” is meant that the nucleotide present in the normal allele is substituted for a different nucleotide in the mutated allele. As used herein, the term “normal allele” refers to the nucleic acid sequence encoding the nonmutated canine von Willebrand's Factor polypeptide. Likewise, the term “mutant allele” refers to the nucleic acid sequence encoding the mutated canine von Willebrand's Factor polypeptide. Specifically, in this embodiment, the “G” present at position 172 of the normal allele of SEQ ID NO:1 is an “A” in the mutant allele.

In another embodiment, the mutation at nucleotide 384 of SEQ ID NO:1 is a nucleic acid deletion mutation. By “nucleic acid deletion mutation” is meant that at least one of the nucleotides present in the normal allele is deleted in the mutant allele. In this embodiment, the “T” at position 384 of SEQ ID NO:2 is deleted.

In another aspect, the invention involves a method of detecting a canine von Willebrand's Factor gene in a sample. This method involves the steps of contacting the sample with an oligonucleotide comprising at least 10 contiguous nucleotides derived from the nucleic acid sequences of SEQ ID NOS: 1 or 2, or complements thereof, and capable of specifically hybridizing with the canine von Willebrand's Factor gene, under conditions favorable for hybridization of the oligonucleotide to any complementary sequence of nucleic acid in the sample and detecting the hybridization, thereby detecting a canine von Willebrand's Factor gene.

In one embodiment, this method further involves the step of quantifying the hybridization of the oligonucleotide to the complementary sequence. In another embodiment, SEQ ID NO:1 has a nucleic acid substitution at nucleotide 172. In a further embodiment, SEQ ID NO:2 has a nucleic acid deletion at nucleotide 384.

In further aspect, the invention involves an assay kit for screening for a canine von Willebrand's Factor gene. This kit contains an oligonucleotide having at least 10 contiguous nucleotides of the nucleic acid sequences of SEQ ID NOS: 1 or 2, or complements thereof, and capable of hybridizing with the nucleotide sequence encoding canine von Willebrand's Factor; reagents for hybridization of the oligonucleotide to a complementary nucleic acid sequence; and container means for the oligonucleotide and the reagents. In various embodiments, SEQ ID NO:1 has a nucleic acid substitution at nucleotide 172 or SEQ ID NO:2 has a nucleic acid deletion at nucleotide 384.

In a different aspect, the invention involves an assay kit for screening for a canine von Willebrand's Factor gene. This kit contains an oligonucleotide having contiguous nucleotides of the nucleic acid sequence that is complementary to the sequence of SEQ ID NO:1 having a mutation at nucleotide 172 and capable of specifically hybridizing to the complementary nucleotide sequence; reagents for the hybridization of the oligonucleotide to a complementary nucleic acid sequence; and container means for the oligonucleotide and the reagents. The mutation in SEQ ID NO:1 is a nucleic acid substitution.

In a still further aspect, the invention involves an assay kit for screening for a canine von Willebrand's Factor gene. This kit contains an oligonucleotide having contiguous nucleotides of the nucleic acid sequence that is complementary to the sequence of SEQ ID NO:2 having a mutation at nucleotide 384 and capable of specifically hybridizing to the complementary nucleotide sequence; reagents for the hybridization of the oligonucleotide to a complementary nucleic acid sequence; and container means for the oligonucleotide and the reagents. The mutation in SEQ ID NO:2 is a nucleic acid deletion mutation.

In yet another aspect, the invention is concerned with methods for detecting a mutated canine von Willebrand's Factor gene in a canine DNA sample. In one embodiment, this method involves the amplification of the DNA sample by polymerase chain reaction to produce polymerase chain reaction products, wherein the polymerase chain reaction uses primers that produce a restriction site in a normal allele but not in a mutant allele, wherein the mutation in the mutant allele is a substitution at nucleotide 172 of the nucleotide sequence encoding canine von Willebrand's Factor polypeptide, wherein the nucleotide sequence is capable of hybridizing under high stringency conditions to the complementary sequence of the sequence of SEQ ID NO:1; digesting the polymerase chain reaction products with a restriction enzyme specific to the restriction site of the primer to produce DNA fragments; and detecting the DNA fragments thereby detecting a mutated canine von Willebrand's Factor gene. The DNA fragments can be detected by, e.g., gel electrophoresis. The primers may alternatively be the primers of SEQ ID NO:3 and SEQ ID NO:4 or the primers of SEQ ID NO:3 and SEQ ID NO:9. In various embodiments, the restriction enzymes may be either Taq I or Hph I.

In another embodiment, this method involves the amplification of the DNA sample by polymerase chain reaction to produce polymerase chain reaction products, wherein the polymerase chain reaction uses primers that are complementary to sequences of the introns flanking the exon, wherein the exon of the mutant allele has a deletion at nucleotide 384 of the nucleotide sequence encoding canine von Willebrand's Factor polypeptide, wherein the nucleotide sequence is capable of hybridizing under high stringency conditions to the complementary sequence of the sequence of SEQ ID NO:2; digesting the polymerase chain reaction products with a restriction enzyme specific to the restriction site of the mutant allele to produce DNA fragments; and detecting the DNA fragments thereby detecting a mutated canine von Willebrand's Factor gene. The DNA fragments can be detected by, e.g., gel electrophoresis. The primers may alternatively comprise the sequence of SEQ ID NO:5 and SEQ ID NO:6 or the sequence of SEQ ID NO:7 and SEQ ID NO:8. In one embodiment, the restriction enzyme is Mwo I.

In another aspect, the invention involves an oligonucleotide probe capable of detecting a mutation associated with canine von Willebrand's disease, wherein the mutation is a base substitution at nucleotide 172 of the nucleotide sequence encoding canine von Willebrand's Factor polypeptide, wherein the nucleotide sequence is capable of hybridizing under high stringency conditions to the complementary sequence of SEQ ID NO:1. In one aspect, the substitution at nucleotide 172 is adenine for guanine.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. Specifically, the polymorphisms are described in U.S. Pat. Nos. 6,074,832 and 6,040,143 as well as WO 00/09533, each of which is incorporated herein by reference. In the case of conflict, the present specification, including definitions, will control. In addition, the materials, methods, and examples are illustrative only and not intended to be limiting.

(Other features and advantages of the invention will be apparent from he following detailed description and claims.

### BRIEF DESCRIPTION OF THE DRAWINGS

The various advantages of the present invention will become apparent to one skilled in the art by reading the following specification and by referencing the following drawings in which:

FIG. 1. is the nucleic acid sequence of part of intron 42, exon 43, and part of intron 43 of the canine von Willebrand's Factor of the present invention (SEQ ID NO: 1). Intronic sequences are shown in lower case and exon 43 is shown in capital letters. Splice donor and acceptor sequences are underlined. The location of the nucleic acid substitution is shown in bold.

FIG. 2 is a comparison of the genomic region encompassing part of intron 42, exon 43, and part of intron 43 (SEQ ID NO:1) with the human von Willebrand's gene located on human chromosome 12 (BAC clone from GenBank). Splice donor and acceptor sequences are underlined.

FIG. 3A illustrates the results of a method of the present invention used to detect mutations in the von Willebrand's gene found in Doberman pinschers, Manchester terriers, Pembroke Welsh corgi's, and Poodles (all three varieties). The forward and reverse primers from introns 42 and 43 are shown (SEQ ID NO: 3 and 4). Splice donor and acceptor sequences are underlined. The nucleic acid substitution is shown in bold.

FIG. 3B shows the result of amplification with the diagnostic primer (SEQ ID NO: 5) of the wild type and mutant allele, resulting in a Taq I restriction site in the wild type, but not the mutant allele.

FIG. 4 is a photograph of an agarose gel showing representative results of the PCR-based diagnostic test for type I von Willebrand's disease as occurs in Doberman pinschers, Manchester terriers, Bernese mountaindogs, Pembroke Welsh corgi's, and Poodles (all three varieties). Lane 1 shows a 100 base pair ladder for size markers. Lane 2 shows Taq I digested DNA of an animal that is homozygous wild type. Lane 3 shows Taq I digested DNA of an animal that is heterozygous wild type (carrier of the mutation). Lane 4 shows Taq I digested DNA of an amimal that is homozygous mutant (affected with von Willebrand's disease).

FIG. 5 is the nucleic acid sequence of part of intron 6, exon 7, and part of intron 7 for the canine von Willebrand's Factor of the present invention (SEQ ID NO: 2). Intronic sequences are given in lower case and exon 7 is shown in upper case. Primer sequences used for amplification (SEQ ID NO: 5 and 6) are indicated by the clear boxes and the naturally occurring Mwo 1 restriction site is indicated by the shaded box

FIG. 6 is a photograph of an agarose gel showing representative results of the PCR-based diagnostic test for type III von Willebrand's disease as occurs in Shetland sheepdogs. Lane 1 shows a 100 base pair ladder for size markers. Lane 2 shows Mwo I digested DNA of an animal that is homozygous wild type. Lane 3 shows Mwo 1 digested DNA of an animal that is heterozygous wild type (carrier of the mutation).

### DETAILED DESCRIPTION OF THE INVENTION

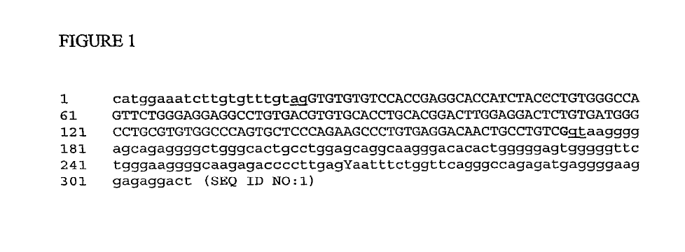

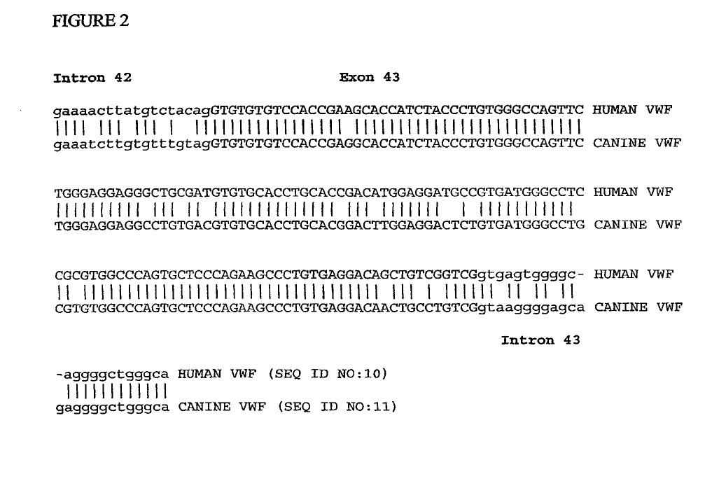

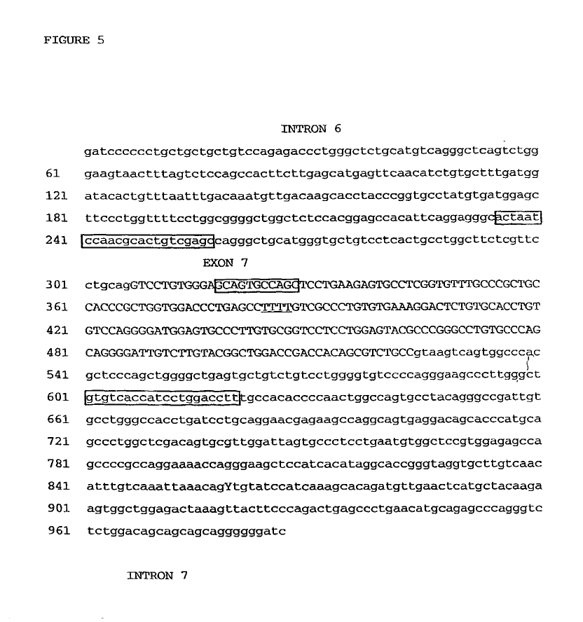

Genomic DNA comprising part of intron 42, all of exon 43, and part of intron 43 from the canine von Willebrand's Factor Gene is set forth in FIG. 1 and SEQ ID NO:1. Genomic DNA comprising part of intron 6, all of exon 7, and part of intron 7 from the canine von Willebrand's Factor gene is set forth in FIG. 5 and SEQ ID NO:2. In one embodiment, the mutation of the normal vWF gene which causes von Willebrand's Disease (vWD) in Shetland Sheepdogs, a single base deletion in exon 7 is provided. In another embodiment, a splice junction mutation at the last nucleotide of exon 43 of the canine von Willebrand's Factor gene, which causes vWD in Doberman pinschers, Manchester terriers and Poodles, and Pembroke welsh corgi's, is provided. The nucleic acid sequences of the present invention may be used in methods for detecting homozygous (mutant or wild type alleles) and heterozygous carriers of the defective vWF gene.

In a preferred method of detecting the presence of the von Willebrand's allele in canines, DNA samples are first collected by relatively non-invasive techniques that do not require penetration into the tissues of the animal. Withdrawing buccal cells via cheek swabs or withdrawing blood samples are two common methods that could be used to obtain DNA (Richards, B. et al., “Multiplex PCR Amplification from the CFTR Gene Using DNA Prepared from Buccal Bushes/Swabs,” Human Molecular Genetics 2:159-163 (1992)). Following isolation of the DNA by standard techniques, PCR is performed on the DNA extracted from these biological samples utilizing primers designed to produce enzyme restriction sites on those DNA samples at with the normal or mutant allele sequence. One skilled in the art will appreciate that this method may be applied not only to Pembroke Welsh corgi's, Doberman pinschers, Shetland sheepdogs, Bernese mountaindogs, Manchester terriers and Poodles, but to other breeds such as Dutch Kooikers (Slappendel, R. J., “von Willebrand's Disease in Dutch Kooiker Dogs,” Vet-Q 17:S21-S22 (1995)), and German Wirehaired pointers as well.

One skilled in the art would appreciate that the presence of the von Willebrand's mutant alleles in canines can also be detected utilizing ligation amplification reaction technology (LAR). LAR is a method analogous to PCR for DNA amplification wherein ligases are employed for elongation in place of polymerases used for PCR. Other methods for allele discrimination in the canine von Willebrand's factor gene, known to those skilled in the art include methods that use allele specific oligonucleotide hybridization, such as molecular beacons (Tyagi S, Kramer F R, “Molecular beacons: probes that fluoresce upon hybridization,” Nat Biotechnol 1996 Mar; 14(3):303-8).

The present invention provides breeders and dog owners with an accurate, definitive test that will provide them with the genotype of their animals with respect to von Willebrand's disease. The information obtained from these genetic tests can, in turn, be used to completely eliminate von Willebrand's disease from breeding populations, by breeding animals that carry the mutation to those that do not have the mutation, and choosing a clear animal for the next generation of breeding. Animals that are currently tested using a protein-based ELISA assay, are often misdiagnosed, as the variability of results with this test results in ambiguity between animals that are carriers and those that are clear. The present invention provides unambiguous diagnosis of von Willebrand's disease in canines as described in the Specific Examples, and methods of the present invention.

It will be appreciated by those skilled in the art, that the present invention provides novel intronic genomic DNA sequences to detect von Willebrand's disease in canines. These sequences have no homology with any other von Willebrand's gene, including humans. Thus, this test is canine specific. The advantage of this is obvious to those skilled in the art, because possible contamination with human DNA will not influence the test results.

The term “gene” as to referred herein means a nucleic acid which encodes a protein product. The term “nucleic acid” refers to a linear array of nucleotides and nucleosides, such as genomic DNA, cDNA, and DNA prepared by partial or total chemical synthesis from nucleotides. The term “encoding” means that the nucleic acid may be transcribed and translated into the desired polypeptide. “Polypeptide” refers to amino acid sequences which comprise both full-length proteins and fragments thereof. “Mutation” as referred to herein includes any alteration in a nucleic acid sequence including, but not limited to, deletions, substitutions and additions.

As referred to herein, the term “capable of hybridizing under high stringency conditions” means annealing a strand of DNA complementary to the DNA of interest under highly stringent conditions. Likewise, “capable of hybridizing under low stringency conditions” refers to annealing a strand of DNA complementary to the DNA of interest under low stringency conditions. In the present invention, hybridizing under either high or low stringency conditions would involve hybridizing a nucleic acid sequence (e.g., the complementary sequence to SEQ ID NO:1 or portion thereof), with a second target nucleic acid sequence. “High stringency conditions” for the annealing process may involve, for example, high temperature and/or low salt content, which disfavor hydrogen bonding contacts among mismatched base pairs. “Low stringency conditions” would involve lower temperature, and/or higher salt concentration than that of high stringency conditions. Such conditions allow for two DNA strands to anneal if substantial, though not nearly complete complementarity exists between the two strands, as is the case among DNA strands that code for the same protein but differ in sequence due to the degeneracy of the genetic code.

Appropriate stringency conditions which promote DNA hybridization, for example, 6×SSC at about 45° C., followed by a wash of 2×SSC at 50° C. are known to those skilled in the art or can be found in Current Protocols in Molecular Biology, John Wiley & Sons, NY (1989), 6.31-6.3.6. For example, the salt concentration in the wash step can be selected from a low stringency of about 2×SSC at 50° C. to a high stringency of about 0.2×SSC at 50° C. In addition, the temperature in the wash step can be increased from low stringency at room temperature, about 22° C., to high stringency conditions, at about 65° C. Other stringency parameters are described in Maniatis, T., et al., Molecular Cloning: A Laboratory Manual, Cold Spring Harbor Laboratory Press, Cold Spring N.Y., (1982), at pp. 387-389; see also Sambrook J. et al., Molecular Cloning: A Laboratory Manual, Second Edition, Volume 2, Cold Spring Harbor Laboratory Press, Cold Spring, N.Y. at pp. 8.46-8.47 (1989).

The invention will be further described in the following examples which do not limit the scope of the invention described in the claims.

### EXAMPLES

### Example 1

Doberman Pinscher, Bernese Mountaindogs, Manchester Terrier, Poodles (all three varieties), and Pembroke Welsh Corgi's.

Materials and Methods

Type 1 von Willebrand's disease is not severe. Affected individuals do have some ability to clot, unlike the more severe Type 3 form of the disease, in which individuals do not produce any vWF protein. As such, one would predict that the mutation for type 1 might be located towards the 3′ end of the gene. Numerous fragments from the 3′ end of the vWF gene including exons 45-47 (data not shown), and an inverse PCR product from exon 43 were cloned and sequenced. Inverse PCR is a technique to obtain DNA sequencing data.

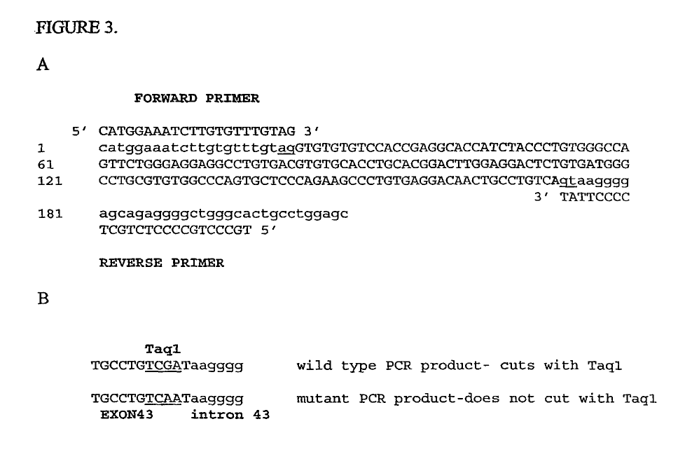

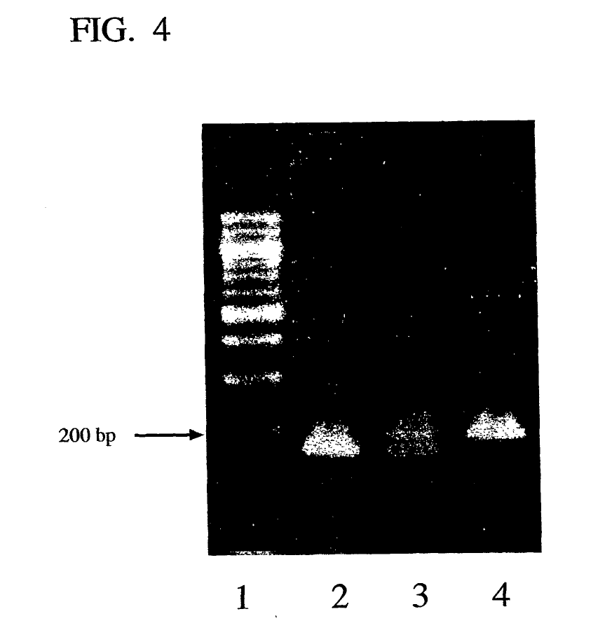

The sequences of the primers used were: intronic 5′ to the mutation (sense primer, forward primer), 5′-GCATGGAAATCTTGTGTTTGTAG-3′ (SEQ ID NO:3); and intronic 3′ to the mutation (antisense primer, reverse primer), 5′-TGCCCTGCCCCTCTGCTCCCCTTAT-3′ (SEQ ID NO: 4). Use of these primers generates a Taq I restriction enzyme site in the wild type sequence, but not the mutant sequence (FIG. 3B). Alternatively, another primer intronic 3′ to the mutation (antisense primer, reverse primer), 5′-TGCCCTGCCCCTCTGCTCCCCTCAC-3′ (SEQ ID NO: 9) is also contemplated. This primer generates a Hph I restriction enzyme site in the wild type sequence, but not the mutant sequence.

PCR-Based Mutation Test. PCR mutagenesis was used to create a Taq I site in the normal allele but not in the mutant allele. The control site is contained within the amplification products of both alleles. The sequences of the primers are: (sense) primer, 5′-GCATGGAAATCTTGTGTTTGTAG-3′ (SEQ ED NO:3) and the diagnostic antisense primer 5′-TGCCCTGCCCCTCTGCTCCCCTTAT-3′ (SEQ ID NO:4). The cycling conditions were 94° C. for two minutes followed by 35 cycles of 92° C. for one minute, 58° C. for one minute 30 sees., and 72° C. for one minute and 30 seconds. 30 μreactions were performed with standard PCR buffer containing 1.5 mM magnesium chloride, except that the PCR enhancer, BSA was added to 0.4 μg/μl. One unit of Taq polymerase was added per reaction Following amplification, 7 μL of buffer containing 25 mM magnesium chloride, and 100 mM NaCl was added. The DNA was digested with 10 units of the restriction enzyme Taq I (New England Biolabs, Beverly Mass.), at 65° C. for one hour, and was then electrophoresed on a 3% agarose gel in 1× Tris-Borate-EDTA (TBE). The running buffer was 1×TBE.

Results

The “A” in the mutant allele found in Doberman pinschers, Poodles, Manchester terriers, and Pembroke welsh corgi's at the end of exon 43 could cause the normal splice junction to be used less frequently, such that the upstream cryptic splice site becomes the one predominantly used. Although this substitution mutation has also been identified in humans, two dogs were heterozygotes and suffered from Type 3 von Willebrand's disease (Zhang Z P, Blomback M, Egberg N, Falk G, Anvret M, 1994, Genomics 21(1):188-93). This mutation was traced to their mother and grandmother, and therefore it was concluded that their severe von Willebrand's disease was inherited from their father. These results show that this is a potential hot spot for mutations in both the canine and human von Willebrand's factor gene. It is possible that this mutation arose independently in all four breeds, rather than from a common ancestor.

The splice junction mutation at the end of exon 43 is the cause of recessive Type 1 vWD found in at least five canine breeds (Doberman pinscher, Bernese mountaindogs, Manchester terrier, Poodle, and Pembroke Welsh corgi's).

### Example 2

Shetland Sheepdog

A mutation was found in the vWF gene that appears to be responsible for most or all of the type 3 vWD found in the Sheltie breed.

A deletion of a single “T” was found at nucleotide position of 384 of SEQ ID NO:2. This deletion, present in the equivalent of human exon 7, would cause a shift in the reading from of the vWF-encoding region, and result in a severely truncated protein.

A diagnostic test was designed to detect this mutation. The deletion of a single “T” in the putative exon 7 of the canine von Willebrand's gene causes the creation of an Mwo I restriction site. the Mwo I site is found in the mutant allele, but not in the normal allele. The sequence is shown in FIG. 5. There is an internal Mwo I site indicated by a shaded box. Standard PCR conditions were used to amplify exon 7 using intronic primer sequences from introns 6 and 7. The forward primer, intronic 5′ to the mutation was 5′-actaatccaacgcactgtcgagc-3′ (SEQ ID NO:5), and the reverse primer, intronic 3′ to the mutation was 5′-aaggtccaggatggtgacac-3′ (SEQ ID NO:6).

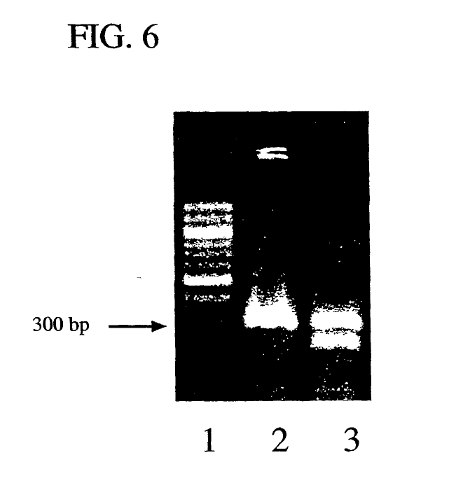

The cycling conditions were: 94° C., 2 min, one cycle, followed by 35 cycles of 94° C., 1 sec, 60° C., 1 minute, and 72° C., one minute, followed by a final extension of 72° C. for three minutes. 30 microliter reactions were performed in duplicate for each animal. Following amplification, 7 μl of buffer containing 25 mM MgCl, and 100 mM NaCl was added the PCR product along with one microliter of Mwo I restriction enzyme (New England Biolabs, Inc.) and incubated at 60° C. for 1 hr. Digestion products were then observed after gel electrophoresis on a 2% agarose gel and the results shown in FIG. 6. Lanes 1 shows a one hundred bp ladder. Lane 2 shows the results from a normal animal, lane 3 shows the results from a carrier animal.

Alternatively, the following DNA sequences to amplify the relevant portion of the vWF gene can also be used: forward primer, intronic 5′ to the mutation 5′-TCACTGCCTGGCTTCTCGTTCCT-3′ (SEQ ID NO:7) and reverse primer, intronic 3′ to the mutation 5′-GGAGCGTGGGCCACTGACTTA-3′ (SEQ ID NO:8).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated nucleic acid molecule comprising a nucleotide sequence encoding mutated canine von Willebrand's Factor polypeptide which causes canine von Willebrand's disease, wherein the nucleotide sequence is capable of hybridizing under high stringency conditions to the complementary sequence of the sequence of SEQ ID NO. 1 having a mutation at nucleotide 172, or SEQ ID NO. 2 having a mutation at nucleotide 384.

2. A vector comprising the nucleic acid molecule of claim 1.

3. A cell comprising the vector of claim 2.

4. The isolated nucleic acid molecule of claim 1, wherein the mutation at nucleotide 172 of SEQ ID NO:1 is a nucleic acid substitution.

5. The isolated nucleic acid molecule of claim 1, wherein the mutation at nucleotide 384 of SEQ ID NO:2 is a nucleic acid deletion.

6. A method of detecting a canine von Willebrand's Factor gene in a sample comprising the steps of:
a) contacting the sample with an oligonucleotide comprising at least 10 contiguous nucleotides derived from the nucleic acid sequence of SEQ ID NO:1 or complement thereof, and capable of specifically hybridizing with the canine von Willebrand's Factor gene, under conditions favorable for hybridization of the oligonucleotide to any complementary sequence of nucleic acid in the sample; and
b) detecting hybridization, thereby detecting a canine von Willebrand's Factor gene.

7. The method of claim 6, further comprising the step of:
c) quantifying hybridization of the oligonucleotide to the complementary sequence.

8. The method of claim 6, wherein SEQ ID NO:1 has a nucleic acid substitution at nucleotide 172.

9. A method of detecting a canine von Willebrand's Factor gene in a sample comprising the steps of:
a) contacting the sample with an oligonucleotide comprising at least 10 contiguous nucleotides derived from the nucleic acid sequence of SEQ ID NO:2 or complement thereof, and capable of specifically hybridizing with the canine von Willebrand's Factor gene, under conditions favorable for hybridization of the oligonuclcotide to any complementary sequence of nucleic acid in the sample; and
b) detecting hybridization, thereby detecting a canine von Willebrand's Factor gene.

10. The method of claim 9, further comprising the step of:
c) quantifying hybridization of the oligonucleotide to the complementary sequence.

11. The method of claim 9, wherein SEQ ID NO:2 has a nucleic acid deletion at nucleotide 384.

12. An assay kit for screening for a canine von Willebrand's Factor gene comprising:
a) an oligonucleotide comprising at least 10 contiguous nucleotides of the nucleic acid sequence of SEQ ID NO:1, and capable of hybridizing with the nucleotide sequence encoding canine von Willebrand's Factor,
b) reagents for hybridization of the oligonucleotide to a complementary nucleic acid sequence; and
c) container means for a) and b).

13. The assay kit of claim 12, wherein SEQ ID NO:1 has a nucleic acid substitution at nucleotide 172.

14. An assay kit for screening for a canine von Willebrand's Factor gene comprising:
a) an oligonucleotide comprising at least 10 contiguous nucleotides of the nucleic acid sequence of SEQ ID NO:2, and capable of hybridizing with the nucleotide sequence encoding canine von Willebrand's Factor;
b) reagents for hybridization of the oligonucleotide to a complementary nucleic acid sequence; and
c) container means for a) and b).

15. The assay kit of claim 14, wherein SEQ ID NO:2 has a nucleic acid deletion at nucleotide 384.

16. An assay kit for screening for a canine von Willebrand's Factor gene comprising:
a) an oligonucleotide comprising contiguous nucleotides of the nucleic acid sequence that is complementary to the sequence of SEQ ID NO:1 having a mutation at nucleotide 172, and capable of specifically hybridizing to the complementary nucleotide sequence;
b) reagents for hybridization of the oligonucleotide to a complementary nucleic acid sequence; and
c) container means for a) and b).

17. The assay kit of claim 16, wherein the mutation at nucleotide 172 is a nucleic acid substitution.

18. An assay kit for screening for a canine von Willebrand's Factor gene comprising:
a) an oligonucleotide comprising contiguous nucleotides of the nucleic acid sequence that is complementary to the sequence of SEQ ID NO:2 having a mutation at nucleotide 384, and capable of specifically hybridizing to the complementary nucleotide sequence;
b) reagents for hybridization of the oligonucleotide to a complementary nucleic acid sequence; and
c) container means for a) and b).

19. The assay kit of claim 18, wherein the mutation at nucleotide 384 is a nucleic acid deletion.

20. A method for detecting a mutated canine von Willebrand's Factor gene in a canine DNA sample comprising the steps of:
a) amplifying the DNA sample by polymerase chain reaction to produce polymerase chain reaction products, wherein the polymerase chain reaction uses primers that produce a restriction site in a normal allele but not in a mutant allele, wherein the mutation in the mutant allele is a substitution at nucleotide 172 of the nucleotide sequence encoding canine von Willebrand's Factor polypeptide, wherein the nucleotide sequence is capable of hybridizing under high stringency conditions to the complementary sequence of the sequence of SEQ ID NO:1;
b) digesting the polymerase chain reaction products with a restriction enzyme specific to the restriction site of the primers to produce DNA fragments; and
c) detecting the DNA fragments, thereby detecting a mutated canine von Willebrand's Factor gene.

21. The method of claim 20, wherein the DNA fragments are detected by gel electrophoresis.

22. The method of claim 20, wherein the primers comprise the sequence of SEQ ID NO:3 and SEQ ID NO:4.

23. The method of claim 22, wherein the restriction enzyme is Taq I.

24. The method of claim 20, wherein the primers comprise the sequence of SEQ ID NO:3 and SEQ ID NO:9.

25. The method of claim 24, wherein the restriction enzyme is Hph I.

26. A method for detecting a mutated canine von Willebrand's Factor gene in a canine DNA sample comprising the steps of:
a) amplifying the DNA sample by polymerase chain reaction to produce polymerase chain reaction products, wherein the polymerase chain reaction uses primers that are complementary to sequences of the introns flanking the exon, wherein the exon of the mutant allele has a deletion at nucleotide 384 of the nucleotide sequence encoding canine von Willebrand's Factor polypeptide, wherein the nucleotide sequence is capable of hybridizing under high stringency conditions to the complementary sequence of the sequence of SEQ ID NO:2;
b) digesting the polymerase chain reaction products with a restriction enzyme specific to the restriction site of the mutant allele to produce DNA fragments; and
c) detecting the DNA fragments, thereby detecting a mutated canine von Willebrand's Factor gene.

27. The method of claim 26, wherein the DNA fragments are detected by gel electrophoresis.

28. The method of claim 26, wherein the primers comprise the sequence of SEQ ID NO:5 and SEQ ID NO:6.

29. The method of claim 26, wherein the primers comprise the sequence of SEQ ID NO:7 and SEQ ID NO:8.

30. The method of claim 28, wherein the restriction enzyme is Mwo I.

31. The method of claim 29, wherein the restriction enzyme is Mwo I.

32. An oligonucleotide probe capable of detecting a mutation associated with canine von Willebrand's disease, wherein the mutation is a base substitution at nucleotide 172 of the nucleotide sequence encoding canine von Willebrand's Factor polypeptide, wherein the nucleotide sequence is capable of hybridizing under high stringency conditions to the complementary sequence of the sequence of SEQ ID NO. 1.

33. The oligonucleotide probe of claim 32, wherein the substitution at nucleotide 172 is adenine for guanine.
